(19) United States
(12) Patent Application Publication
(10) Pub. No.: US 2005/0053567 A1
(43) Pub. Date: Mar. 10, 2005

(54) Aqueous cosmetic composition

(76) Inventor: Yingchun Liu, Guelph (CA)

Correspondence Address: Yingchun Liu, 85 Porter Court, Guelph, ON, N1L 1L8, CA

(21) Appl. No.: 10/378,059

(22) Filed: Mar. 4, 2003

(51) Int. Cl. A61K 7/06; A61K 7/11

(52) U.S. Cl. 424/70.16

(57) ABSTRACT

The invention provides an aqueous cosmetic composition for providing a durable crosslinked film on a keratinous substrate having at least two water-based film-form polymers including at least one water-based self-crosslinking film-form polymer. The self-crosslinking film-form polymer has functional groups capable of crosslinking with each other after the cosmetic composition is placed on the keratinous substrate. The aqueous cosmetic composition as whole forms a film on keratinous substrate that adheres to the keratinous substrate and is removable and durable.

This application is a continuation of Provisional Patent Application Ser. No. 60/359,715, filed Feb. 27, 2002.

### FIELD OF THE INVENTION

This invention relates to cosmetic compositions and, more particularly, an aqueous cosmetic composition, for providing a durable yet removable crosslinked film on a keratinous substrate, comprising at least two water-based film-form polymers including at least one water-based self-crosslinking film-form polymer.

### BACKGROUND OF THE INVENTION

In order for cosmetic compositions to perform satisfactorily, the cosmetic compositions must exhibit a number of desirable performance properties. Cosmetic compositions are known which provide films on keratinous substrates, and which have desirable performance properties. For example, the film created on a keratinous substrate by the cosmetic composition must adhere to the keratinous substrate, exhibits desired gloss, can be easily removed, and it also should be durable. In other words, desirable performance properties for a cosmetic composition that provides a film on a keratinous substrate would include adhesion, good removability, appearance and durability.

In general, however, the known cosmetic compositions, particularly in the case of nail polishes, exhibit desirable performance properties are not aqueous. Most of the nail polishes on the market today are formulated undesirably with organic solvents such as ethyl acetate, butyl acetate, methyl ethyl ketone, and toluene. These organic solvents are undesirable because they tend to be flammable and adversely affect air quality. Organic solvents also tend to discolor the nails, and make them brittle. Because of these problems, cosmetic scientists have investigated developing aqueous nail coating compositions.

Durability and adhesion are the major challenges when formulating aqueous nail polishes. Many approaches have involved using a blend of aqueous dispersions of film-form polymers. Using a blend of film-form polymers in cosmetic composition rather than using a single film-form polymer gives the cosmetic formulators much better opportunity to get the desired performance properties of the cosmetic composition, such as flexibility, adhesion, durability and so on. U.S. Pat. No. 5,601,808 discloses an aqueous nail coating composition having a blend of aqueous dispersions including at least one polyurethane polymer. However, the nail coating is lack of adhesion to nails and is thus described as “peel off” nail coating.

To obtain desired durability and adhesion for aqueous nail coatings, using aqueous self-crosslinking film-form polymer is one of the approaches. U.S. Pat. No. 6,277,358 B1 (Calello et al.) and U.S. Patent Application 20020004054 (Calello et al.) disclose an aqueous cosmetic composition which contains self-crosslinking film-form polymer. It also discloses an aqueous cosmetic composition in which a blend of a self-crosslinking film-form polymer and a limited amount of cellulose-based film formers are used. However, it has been found that determining a self-crosslinking film-form polymer, which will enable the cosmetic composition to have the desired performance properties, is very difficult. It has also been found that determining a blend of a self-crosslinking film-form polymer and a limited amount of cellulose-based film former, which will enable the cosmetic composition to have the desired performance properties, is also very difficult. Also cellulose-based film-form polymer has disadvantages, for example, nail coating formulations containing nitrocellulose tend to discolor the nails and over time tend to drop in viscosity and lose the ability to form a hard film.

U.S. Pat. No. 6,277,358B1 and U.S Patent Application 20020004054 did not disclose that by blending self-crosslinking film-form polymer with thermoplastic film-form polymer other than cellulose-based thermoplastic film-form polymer, an aqueous cosmetic composition, particularly aqueous nail coating composition can be produced which has particularly much improved removability. There are many other advantages by employing this technology to formulate aqueous cosmetic compositions, such as improved flexibility and durability, reduced amount of coalescing solvents, reduced amount of plasticizers and so on.

There is therefore a need for an aqueous cosmetic composition for providing a film on a keratinous substrate that is durable and easily removable.

### SUMMARY OF THE INVENTION

In a broad aspect of the present invention, there is provided an aqueous cosmetic composition for providing a durable crosslinked film on a keratinous substrate, having at least two water-based film-form polymers, including at least one self-crosslinking film-form polymer. The self-crosslinking film-form polymer has functional groups capable of crosslinking with each other after the cosmetic composition is placed on the keratinous substrate. In addition, the other water-based film-form polymers are for modifying the performance properties of the water-based self-crosslinking film-form polymer. Accordingly, the other water-based film-form polymer has performance properties that differ substantially from the performance properties of the self-crosslinking film-form polymer. In particular, the performance properties of the self-crosslinking film-form polymer are modified so that the crosslinked film formed on the keratinous substrate by the cosmetic composition adheres to the keratinous substrate and is removable, durable and exhibit desired gloss. The other water-based film-form polymer should also be compatible with the self-crosslinking film-form polymer meaning it forms a clear film together with the self-crosslinking film-form polymer under desired film-forming condition, and offer the final aqueous cosmetic composition sufficient shelf life.

In another aspect of the invention, the other water-based film-form polymers are all water-based thermoplastic film-form polymers.

In yet another aspect of the present invention, the other water-based film-form polymers including at least another water-based self-crosslinking film-form polymer.

The self-crosslinking film-form polymer, as described above, does not require external crosslinking agent for crosslinking. It has built-in functional groups that are capable of crosslinking with each other after the cosmetic composition is applied on to a keratinous substrate. It thus forms a one component crosslinking system. The crosslinking reaction only takes place after the cosmetic composition is placed on a keratinous substrate. When the self-crosslinking film-form polymer is in liquid state before it is applied on to a keratinous substrate, no crosslinking reaction takes place since at least one of the functional groups for crosslinking is being blocked by a blocking agent. Once the self-crosslinking polymer is applied on to a keratinous substrate, the blocking agent dissipates, the functional group for crosslinking once was blocked, becomes available for crosslinking.

### DETAILED DESCRIPTION OF THE PREFERRED EMBODIMENT(S)

In a preferred embodiment of the present invention, an aqueous cosmetic composition for providing a crosslinked film on a keratinous substrate has at least one water-based self-crosslinking film-form polymer and at least another water-based film-form polymer. Accordingly, the other water-based film-form polymer has performance properties, which differ substantially from the first self-crosslinking film-form polymer, so that the crosslinked film provided on the keratinous substrate has the desired performance properties as described above. The first water-based self-crosslinking film-form polymer has functional groups capable of crosslinking with each other after the cosmetic composition is placed on the keratinous substrate.

In a preferred embodiment, the second water-based film-form polymer is a water-based thermoplastic film-form polymer. It is preferred that the first water-based self-crosslinking film-form polymer is at least one first compound selected from the group consisting of acrylic copolymer, acrylic-methacrylate copolymer, styrene-acrylic copolymer, acrylonitrile-acrylic copolymer, urethane-acrylic hybrid, polyurethane polymer, vinyl copolymer, vinyl acetate-acrylic copolymer and polyacrylate/polysilicate hybrid. It is also preferred that the water-based thermoplastic film-form polymer is at least one second compound selected from the group consisting of acrylic copolymer, acrylic-methacrylate copolymer, styrene-acrylic copolymer, polyurethane polymer, vinyl copolymer, vinyl acetate-acrylic copolymer, polysaccharide water dispersion, acyrlonitrile-acrylate copolymer, polyester, polyamide, polyimide, and polyvinylalcohol.

As can be seen from the foregoing paragraph, in this embodiment, a wide variety of compounds may be employed as the first water-based self-crosslinking film-form polymer or the water-based thermoplastic film-form polymer. Furthermore, mixtures of various compounds may be used as the first water-based self-crosslinking film-form polymer or the water-based thermoplastic film-form polymer. As noted above, however, the performance properties of the water-based thermoplastic film-form polymer differ substantially from the performance properties of the first water-based self-crosslinking film-form polymer. The water-based thermoplastic film-form polymer should also be compatible with the first self-crosslinking film-form polymer meaning it forms a clear film together with the self-crosslinking film-form polymer under desired film-forming condition, and offer the final aqueous cosmetic composition sufficient shelf life.

Preferably, the aqueous cosmetic composition comprises about, on the polymer solid basis, 1 to about 80 percent by weight the first water-based self-crosslinking film-form polymer and about 1 to about 80 percent by weight the water-based thermoplastic film-form polymer. It is also preferred that the aqueous cosmetic composition comprises about, again on the polymer solid basis, 10 to about 50 percent by weight the first water-based self-crosslinking film-form polymer and about 10 to about 50 percent by weight the water-based thermoplastic film-form polymer. It is also preferred that, in the aqueous cosmetic composition, the ratio of the first water-based self-crosslinking film-form polymer to the water-based thermoplastic film-form polymer is between about 0.1:99.9 and about 99.9:0.1 on a polymer solid basis.

In an alternative embodiment, the other water-based film-form polymer is a second water-based self-crosslinking film-form polymer. It is preferred that the first water-based self-crosslinking film-form polymer is at least one first compound selected from the group consisting of acrylic copolymer, acrylic-methacrylate copolymer, styrene-acrylic copolymer, acrylonitrile-acrylate copolymer, urethane-acrylate hybrid, polyurethane polymer, vinyl copolymer, and polyacrylate/polysilicate hybrid. It is also preferred that the second water-based self-crosslinking film-form polymer is at least one second compound selected from the group consisting of acrylic copolymer, acrylic-methacrylate copolymer, styrene-acrylic copolymer, acrylonitrile-acrylate copolymer, urethane-acrylate hybrid, polyurethane polymer, vinyl copolymer, and polyacrylate/polysilicate hybrid.

As can be seen from the foregoing paragraph, a wide variety of compounds may be employed as the first water-based self-crosslinking film-form polymer or the second water-based self-crosslinking film-form polymer. Furthermore, mixtures of various compounds may be used as the first water-based self-crosslinking film-form polymer or the second water-based self-crosslinking film-form polymer. As noted above, however, the performance properties of the second water-based self-crosslinking film-form polymer differ substantially from the performance properties of the first water-based self-crosslinking film-form polymer. The second water-based self-crosslinking film-form polymer should also be compatible with the first self-crosslinking film-form polymer meaning it forms a clear film together with the first self-crosslinking film-form polymer under desired film-forming condition, and offer the final aqueous cosmetic composition sufficient shelf life.

Among the desirable performance properties are adhesion, appearance, removability, and durability. The aqueous cosmetic composition forms a crosslinked film on the keratinous substrate which should adhere to the substrate. Also, the crosslinked film should be relatively easily removable. Finally, the crosslinked film should be relatively durable and flexible, rather than brittle or rigid, so that the crosslinked film tends not to break or crack while in place, adhering to the keratinous substrate.

Preferably, the aqueous cosmetic composition comprises about, on the polymer solid basis, 1 to about 80 percent by weight the first water-based self-crosslinking film-form polymer and about 1 to about 80 percent by weight the second water-based self-crosslinking film-form polymer. It is also preferred that the aqueous cosmetic composition comprises about, on the polymer solid basis, 10 to about 50 percent by weight the first water-based self-crosslinking film-form polymer and about 10 to about 50 percent by weight the second water-based self-crosslinking film-form polymer. It is also preferred that, in the aqueous cosmetic composition, the ratio of the first water-based self-crosslinking film-form polymer to the second water-based self-crosslinking film-form polymer is between about 0.1:99.9 and about 99.9:0.1 on a polymer solid basis.

It will be evident to those skilled in the art that the invention can take many forms, and that such forms are within the scope of the invention as claimed. The spirit and scope of the appended claims therefore should not be limited to the description of the preferred versions contained herein.

### EXAMPLES

The following examples further describe and demonstrate embodiments within the scope of the present invention. The examples are given solely for the purpose of illustration and are not to be constructed as limitations of the present invention, as many variations thereof are possible without departing from the spirit and scope of the invention.

### Example 1

An aqueous nail coating composition in accordance with the invention incorporates a blend of water-based self-crosslinking acrylic film-form polymer and a thermoplastic water-based film-forming polymer was made according to the following formula:

| | w/w % | |
|---|---|---|
| Neocryl XK14 | 45.75 | water-based self-crosslinking film-form |
| | | polymer* |
| Neocryl A1049 | 45.75 | thermoplastic water-based film-form |
| | | polymer** |
| Premix: | | |
| Texanol | 4.0 | coalescent/film-forming aid*** |
| Water | 4.5 | |

Add premix to the batch slowly while mixing
*manufactured by NeoResins, Avecia Inc., Wilmington, MA USA
**” manufactured by SC Johnson Polymer, Sturtewvant, WI, USA

### Example 2

An aqueous nail coating composition in accordance with the invention incorporates a blend of water-based self-crossking urethane film-form polymer and a thermoplastic water-based film-form polymer was made according to the following formula:

| | w/w % | |
|---|---|---|
| Witcobond W240 | 49.5 | waterbased self-crosslinking urethane |
| | | film-form polymer* |
| Nacrylic 6408 | 33.0 | thermoplastic acrylic water-based film-form |
| | | polymer** |
| Premix: | | |
| Texanol | 7.0 | coalescent/film-forming aid |
| Water | 10.5 | |

Add premix to the batch slowly while mixing
*Manufactured by Uniroyal Chemicals, Elmira, Ontario, Canada
**manufactured by Nacan Products Ltd., Brampton, Ontario, Canada

### Example 3

An aqueous nail coating composition in accordance with the invention incorporates a blend of two water-based self-crosslinking acrylic film-form polymers was made according to the following formula:

| | w/w % | |
|---|---|---|
| Neocryl XK14 | 45.75 | water-based self-crosslinking film-form |
| | | polymer |
| Witcobond W240 | 45.75 | water-based self-crosslinking film-form |
| | | polymer |
| Premix: | | |
| Texanol | 4.0 | coalescent/film-forming aid |
| Water | 4.5 | |

Add premix to the batch slowly while mixing

1. An aqueous cosmetic composition for application to keratinous substrate, comprising at least two water-based film-form polymers including at least one water-based self-crosslinking film-form polymer and at least another water-based film-form polymer, wherein said self-crosslinking film-form polymer having functional groups capable of crosslinking after the said aqueous cosmetic composition is placed on a keratinous substrate.

2. The said self-crosslinking film-form polymer according to claim 1 comprising: (a) at least two different functional groups capable of crosslinking with each other after said aqueous cosmetic composition is applied on to a keratinous substrate, (b) at least one of the said different functional groups of the said self-crosslinking film form polymer is blocked from crosslinking before the said aqueous cosmetic composition is placed on to a keratinous substrate, (c) an dissipatable blocking agent which (i) is operable prior to dissipation, to block at least one of the said functional groups of the said self-crosslinking polymer, and which (ii) after dissipation, leaves the functional group free for crosslinking.

3. An aqueous cosmetic composition according to claim 1 wherein said at least another water-based film-form polymer is thermoplastic film-form polymer.

4. An aqueous cosmetic composition according to claim 3 the weight ratio of total said self-crosslinking film-form polymer(s) to total said thermoplastic film-form polymer(s) is between about 0.1:99.9 and about 99.9:0.1 on polymer solid basis.

5. An aqueous cosmetic composition as claimed in claim 3 wherein said water-based self-crosslinking film-form polymer is selected from the group consisting of acrylic copolymer, acrylic-methacrylate copolymer, styrene-acrylic copolymer, acrylonitrile-acrylate copolymer, urethane-acrylate hybrid polymer, polyurethane polymer, vinylacetate-acrylate copolymer, polyacrylate/polysilicate hybrid and combinations thereof, and the said water-based thermoplastic film-form polymer is selected from the group consisting of acrylate copolymer, acrylate-methacrylate copolymer, styrene-acrylate copolymer, acrylonitrile-acrylate copolymer, polyurethane-polyacrylate hybrid, polyurethane polymer, vinylacetate/acrylate copolymer, polyacrylate/polysilicate hybrid, polysaccharide polymer, cellulosic film-forming polymer and combinations thereof.

6. The said keratinous substrate according to claim 3 includes nails, hair and skin.

7. The composition of claim 3 which is a nail coating composition comprising, on the polymer solid basis, 1-90% by weight of the total composition of total said film-form polymers.

8. The composition of claim 7 also includes one or more compositions selected from the group consisting essentially of plasticizing agents, coalescing agents, antifoaming agents, viscosifying agents, suspending agents, leveling aids, waxes and pigments.

9. A method of coating nails comprising applying the composition of claim 7 onto one or more nails.

10. An aqueous cosmetic composition for keratinous substrate as claimed in claim 1 wherein said at least another film-form polymer is a second self-crosslinking film-form polymer.

11. An aqueous cosmetic composition as claimed in claim 10 in which the first water-based self-crosslinking film-form polymer is at least one first compound selected from the group consisting of acrylic copolymer, acrylic-methacrylate copolymer, styrene-acrylic copolymer, acrylonitrile-acrylate copolymer, urethane-acrylate hybrid, polyurethane polymer, vinyl copolymer, and polyacrylate/polysilicate hybrid, and combinations thereof, and the said a second water-based self-crosslinking film-form polymer is at least one second compound selected from the group consisting of acrylic copolymer, acrylic-methacrylate copolymer, styrene-acrylic copolymer, acrylonitrile-acrylate copolymer, urethane-acrylic hybrid, polyurethane polymer, vinyl copolymer, and polyacrylate/polysilicate hybrid and combinations thereof.

12. The composition of claim 10 which is a nail coating composition comprising on the polymer solid basis, 1-90% by weight of the total composition of total said film-form polymers.

13. An aqueous cosmetic composition according to claim 10 the weight ratio of said first self-crosslinking film-form polymer(s) to the said second self-crosslinking film-form polymer(s) is between about 0.1:99.9 and about 99.9:0.1 on polymer solid basis

14. The composition of claim 12 also includes one or more compositions selected from the group consisting essentially of plasticizing agents, coalescing agents, antifoaming agents, viscosifying agents, suspending agents, leveling aids, waxes and pigments.

15. The said keratinous substrate according to claim 10 includes nails, hair and skin.

16. A method of coating nails comprising applying the composition of claim 12 onto one or more nails.
